(19) United States
(12) Patent Application Publication
(10) Pub. No.: US 2010/0210891 A1
(43) Pub. Date: Aug. 19, 2010

(54) BRACHYTHERAPY TREATMENT DEVICE

(76) Inventors: Darrell Drysen, Altadena, CA (US); Fredrick W. Wintch, Snohomish, WA (US)

Correspondence Address: MCDERMOTT WILL & EMERY LLP, 2049 CENTURY PARK EAST, 38th Floor, LOS ANGELES, CA, 90067-3208, US

(21) Appl. No.: 12/606,971

(22) Filed: Oct. 27, 2009

Related U.S. Application Data
(60) Provisional application No. 61/114,903, filed on Nov. 14, 2008.

(51) Int. Cl. A61M 36/12

(52) U.S. Cl. 600/7

(57) ABSTRACT

Brachytherapy devices and methods that may allow high doses of radiation to be applied to areas in need of treatment, while reducing damage to healthy tissue that may be in the vicinity. The devices and methods may utilize an expansion device and a treatment device. The expansion device may include an outer expandable assembly that is implanted into a post-surgical cavity. The treatment device may include an inner expandable assembly that is mated to the expansion device during treatment fractions, and provides a pathway for the radiation source, for example an HDR (high dose rate) afterloader source.

### CROSS-REFERENCE TO RELATED APPLICATIONS

This application is based on and claims priority to U.S. Provisional Application Ser. No. 61/114,903, entitled “Brachytherapy Treatment Device,” Attorney Docket No. 085310-1017 (063344-0085), filed Nov. 14, 2008.

This application is also related to U.S. application Ser. No. 12/544,101, entitled “Expandable Brachytherapy Device with Constant Radiation Source Spacing,” Attorney Docket No. 085310-0011, filed Aug. 19, 2009, which is a continuation of U.S. application Ser. No. 12/037,534, entitled “Expandable Brachytherapy Device with Constant Radiation Source Spacing,” Attorney Docket No. 085310-1011, filed Feb. 26, 2008; which is a continuation of U.S. application Ser. No. 11/737,028 (now U.S. Pat. No. 7,357,770), entitled “Expandable Brachytherapy Device With Constant Spacing,” Attorney Docket No. 085310-0015, issued Apr. 15, 2008; which claims the benefit of U.S. Provisional Application Ser. No. 60/864,288, entitled “Brachytherapy Device Having Seed Tubes With Individually-Settable Tissue Spacings,” Attorney Docket No. 63344-045, filed Nov. 3, 2006.

This application is also related to U.S. patent application Ser. No. 12/137,437, entitled “Expandable Brachytherapy Device with Constant Spacing,” Attorney Docket No. 085310-0018, filed Jun. 11, 2008; which is a Continuation-in-Part of U.S. application Ser. No. 11/737,028 (now U.S. Pat. No. 7,357,770), referenced above.

This application is also related to U.S. application Ser. No. 12/554,732, entitled “Brachytherapy Apparatus for Asymmetrical Body Cavities,” Attorney Docket No. 085310-0012, filed Sep. 4, 2009; which is a continuation of U.S. application Ser. No. 11/379,739, entitled “Brachytherapy Apparatus for Asymmetrical Body Cavities,” Attorney Docket No. 085310-1012, filed Apr. 21, 2006; which is a Continuation-in-Part of U.S. patent application Ser. No. 11/305,437, entitled “Brachytherapy Apparatus,” Attorney Docket No. 085310-0014, filed Dec. 16, 2005.

This application is also related to U.S. application Ser. No. 12/557,040, entitled “Brachytherapy Device Having an Alignment and Seal Adaptor,” Attorney Docket No. 085310-0013, filed Aug. 19, 2009; which is a Continuation of U.S. application Ser. No. 11/741,670, entitled “Brachytherapy Device Having an Alignment and Seal Adaptor,” Attorney Docket No. 085310-1013, filed Apr. 27, 2007.

This application is also related to U.S. application Ser. No. 11/935,348, entitled “Brachytherapy Device Having Seed Tubes with Individually Settable Tissue Spacings,” Attorney Docket No. 085310-0016, filed Nov. 5, 2007.

All of these applications are incorporated herein by reference.

### BACKGROUND

1. Field

This application relates to brachytherapy.

2. Description of Related Art

Brachytherapy is used in a variety of treatments. Often times, a high dose of radiation is needed. However, it may be difficult to apply a high dose to areas in need of treatment, without also causing damage to healthy tissue in the vicinity.

### SUMMARY

Brachytherapy devices and methods are disclosed that may allow high doses of radiation to be applied to areas in need of treatment, while reducing damage to healthy tissue that may be in the vicinity.

The devices and methods may utilize an expansion device and a treatment device. The expansion device may include an outer expandable assembly that is implanted into a post-surgical cavity. The treatment device may include an inner expandable assembly that is mated to the expansion device during treatment fractions, and provides a pathway for the radiation source, for example an HDR (high dose rate) afterloader source.

The outer expandable assembly in the expansion device may be inserted into a post-surgical cavity (for example, a post-lumpectomy cavity), and deployed to hold the cavity open. The expansion device may include a locking feature that maintains deployment, and an indexing feature. The indexing feature may mate the treatment device with the expansion device in such away that a consistent orientation between the devices is maintained during each treatment fraction.

The expansion device may further include a membrane barrier or expandable sheath that separates the treatment device from the post-surgical cavity, and protects the treatment device from bodily fluids and other contaminants.

The devices and methods may utilize a set of bendable tubes or lumens. At least some of the tubes may be bowed to fill the post-surgical cavity, and thus function as outer tubes in the outer expandable assembly of the expansion device. Other tubes may be located within the volume created by the bowed outer tubes, protected by the sheath, and thus function as inner lumens in the inner expandable assembly in the treatment device. A radiation source, such as one or more radiation seeds, may be placed within one or more of the inner lumens.

The inner lumens in the treatment device may interface with an HDR afterloader to guide one or more treatment source(s) into the post-surgical cavity. The treatment device may interact with the expansion device by means of the indexing feature which allows constant and repeatable orientation of the treatment device within the cavity. The inner lumens may be expanded and contracted by means of an actuator on the device, allowing for easy insertion and removal between dose fractions. The inner lumens may be adjusted individually for greater dose conformality.

These, as well as other components, steps, features, objects, benefits, and advantages, will now become clear from a review of the following detailed description of illustrative embodiments and the accompanying drawings.

### BRIEF DESCRIPTION OF DRAWINGS

The drawings disclose illustrative embodiments. They do not set forth all embodiments. Other embodiments may be used in addition or instead. Details that may be apparent or that are unnecessary are also often omitted to save space or for more effective illustration. When the same numeral appears in different drawings, it is intended to refer to the same or like components or steps.

FIG. 1 illustrates an expansion device installed in a post-lumpectomy cavity, and a treatment device that is removed from the expansion device.

FIG. 2 illustrates the treatment device being locked into the expansion device for the duration of a treatment fraction.

FIG. 3 illustrates various components of the expansion device.

FIG. 4 illustrates a deployment mechanism for the expansion device that utilizes ratchet teeth.

FIG. 5 illustrates another deployment mechanism for the expansion device that utilizes a screw thread.

FIG. 6 illustrates various components of the treatment device.

FIG. 7 illustrates an actuator mechanism for expanding and contracting the inner lumens in the treatment device.

FIG. 8 illustrates in more detail a pin and slot used in the actuator mechanism shown in FIG. 7.

FIG. 9 illustrates a keying feature for mating the treatment device to the expansion device so as to achieve repeatable orientation.

### DETAILED DESCRIPTION OF ILLUSTRATIVE EMBODIMENTS

Illustrative embodiments are now discussed. Other embodiments may be used in addition or instead. Details that may be apparent or that are unnecessary are also often omitted to save space or for more effective presentation.

A brachytherapy device that includes an expandable outer cage and an expandable inner cage positioned within the outer cage is described in U.S. Pat. No. 7,357,770, entitled “Expandable Brachytherapy Device With Constant Radiation Source Spacing,” issued Apr. 15, 2008, the entire content of which is incorporated herein by reference.

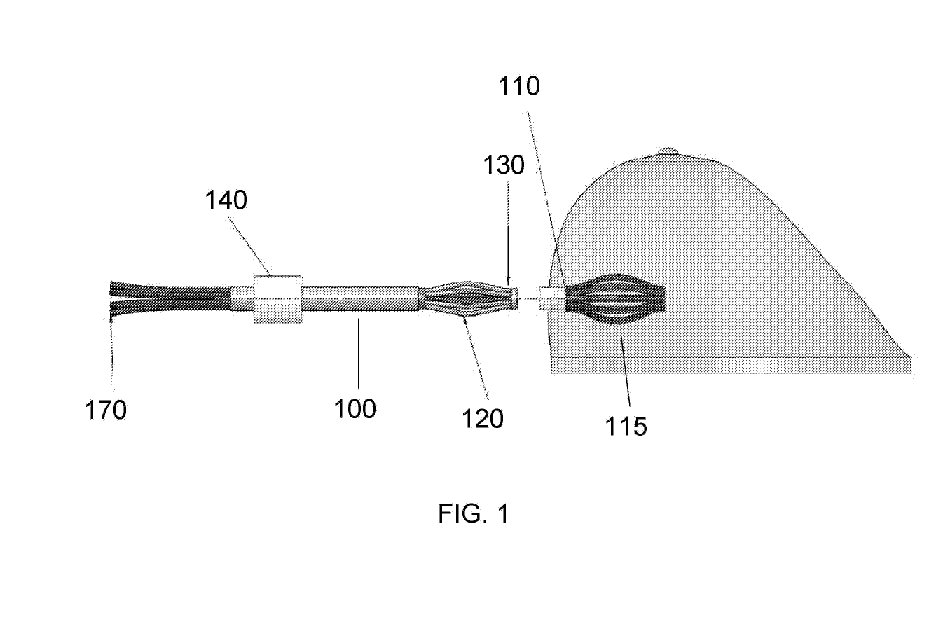

FIG. 1 illustrates an expansion device 110 that is shown as having been installed inside a post-lumpectomy cavity, and a treatment device 100 that is shown as having been removed from the expansion device 110. As seen in FIG. 1, the treatment device 100 is completely separable from the expansion device 110: the treatment device 100 is insertable within the expansion device 110 during radiation treatment, and removable from the expansion device 110 after the treatment.

The expansion device 110 includes an outer expandable assembly 115, which is shown in FIG. 1 in an expanded state and as having been inserted within a cavity in a breast. The cavity may be formed by the extraction of a cancerous tumor or may exist for any other reason. The treatment device 100 includes an inner expandable assembly 120, which is positionable within the outer expandable assembly 115, and which is shown in FIG. 1 in a partially expanded state.

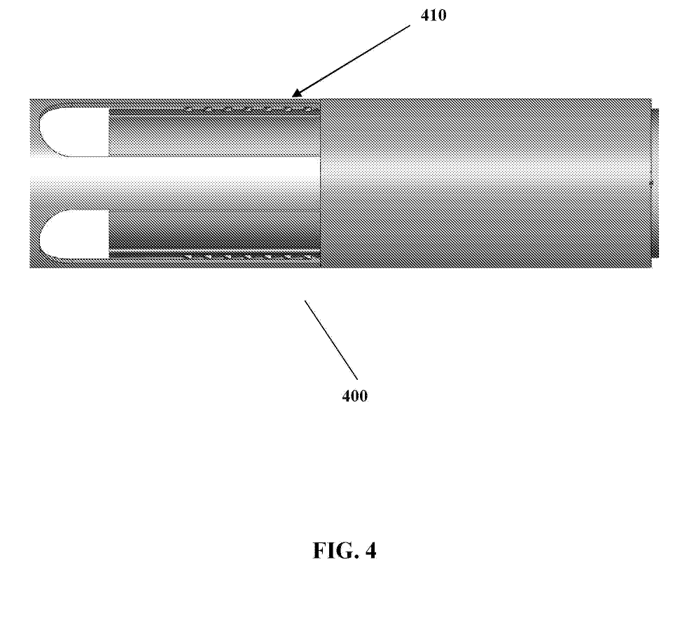

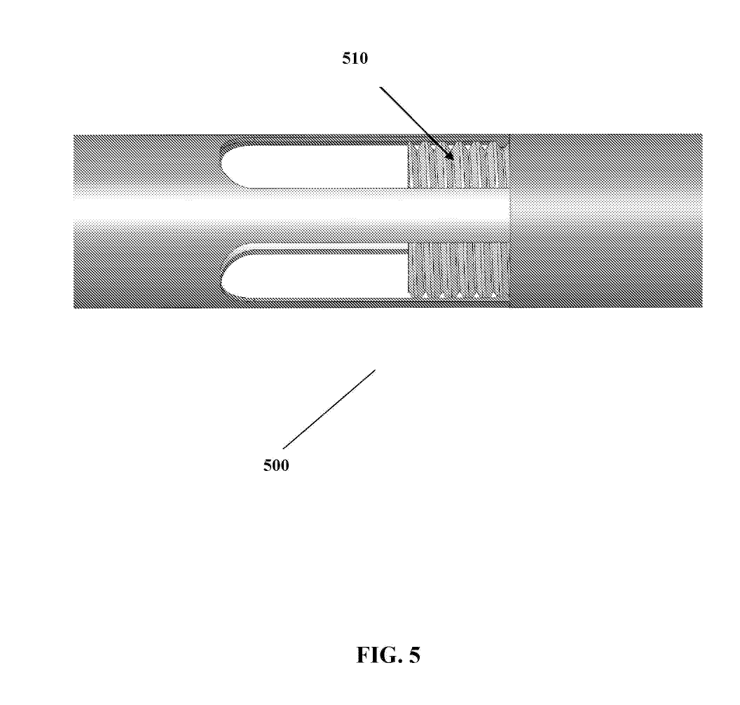

The outer assembly 115 may be expanded or deployed, then held in place, by a deployment mechanism that is described further in conjunction with FIG. 4 and FIG. 5 below. As shown in FIG. 1, the outer expandable assembly 115 may include a plurality of outer tubes.

The inner expandable assembly 120 may include inner tubes or lumens, each of which may be configured to receive radioactive material at different locations therein. For example, the inner lumens may be connectable to an HDR afterloader through connections 170.

A movable actuator 140, further described in conjunction with FIG. 4 below, may be configured to cause the inner assembly 120 to expand in response to movement of the actuator.

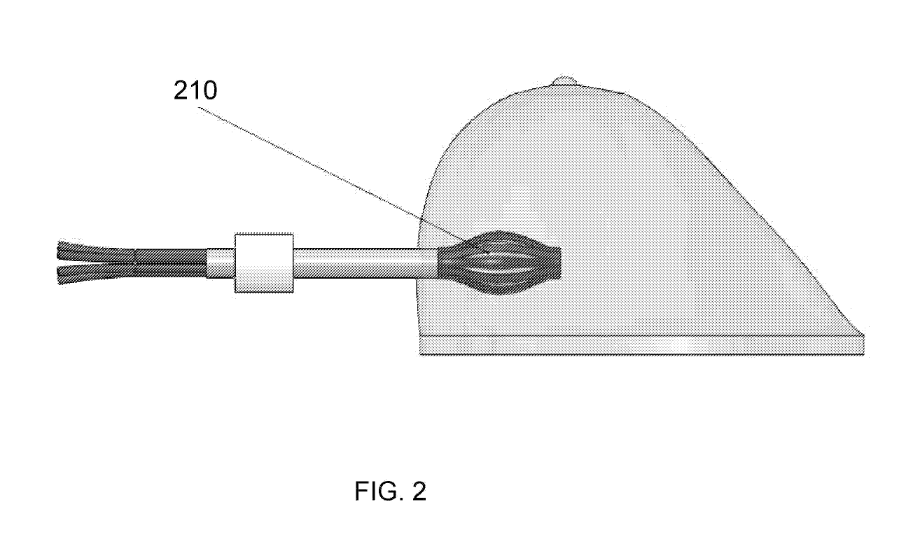

FIG. 2 illustrates a treatment device 210 that is locked into the expansion device for the duration of a treatment fraction. The expansion device (including the outer assembly) is inserted in the cavity and expanded first, after which the treatment device (including the inner assembly) is inserted into the interior volume created by the expanded outer assembly. The outer assembly is usually expanded to fill the size of the cavity. The inner assembly is then expanded according to the prescription of the oncologist. Throughout the radiation treatment or treatment fraction, the inner assembly 210 remains completely nested within the expanded and deployed outer assembly.

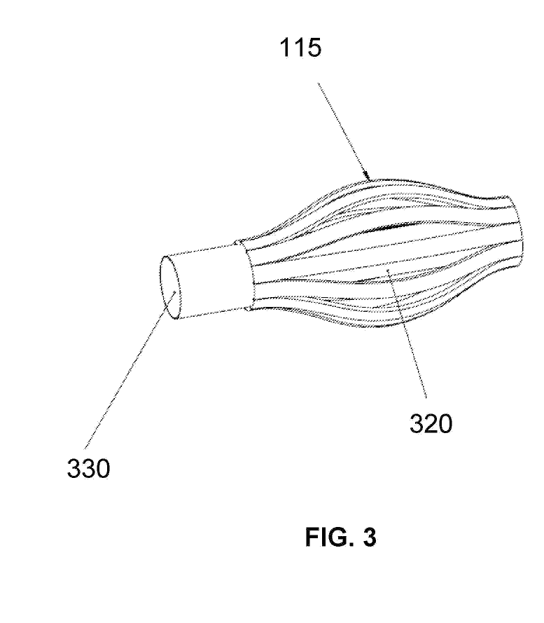

FIG. 3 illustrates in more detail various components of the expansion device. As shown in FIG. 3, the expansion device includes an outer expandable assembly or cage 310, which may be expanded and contracted by an actuator, further described in conjunction figure FIGS. 4 and 5 below. The outer expandable assembly 310 may include a plurality of flexible outer tubes. The outer tubes may define an interior volume in which the inner tubes of the inner expandable assembly 115 (shown in FIG. 1) may reside when inserted within the cavity. Each of the inner tubes and each of the outer tubes may be hollow, and thus configured to receive a radioactive seed or other therapeutic element therewithin. In an alternate embodiment, one or more of the inner and/or the outer tubes may not be hollow, but rather solid, opaque, or otherwise unable to receive a therapeutic element therewithin.

The expansion device may include an inner expandable sheath or membrane 320, which is configured to cover and seal the inner expandable assembly when the inner assembly is inserted within the expanded and deployed outer assembly 310. When the outer assembly 310 is fully inflated and deployed, the expandable sheath 320 may provide a dry protected interior region for the inner assembly, when inserted within the deployed outer assembly 310. The expandable sheath 320 shields the inner assembly from bodily fluids and other contaminants in the cavity. When the treatment device is withdrawn from the post-surgical cavity, the sheath 320 remains in the interior of the expansion device.

The expandable sheath or membrane 320 may be made of a variety of polymer materials, including but not limited to polyurethane, PVC, PVB, neoprene, polystyrene, polyacrylonitrile, and silicone.

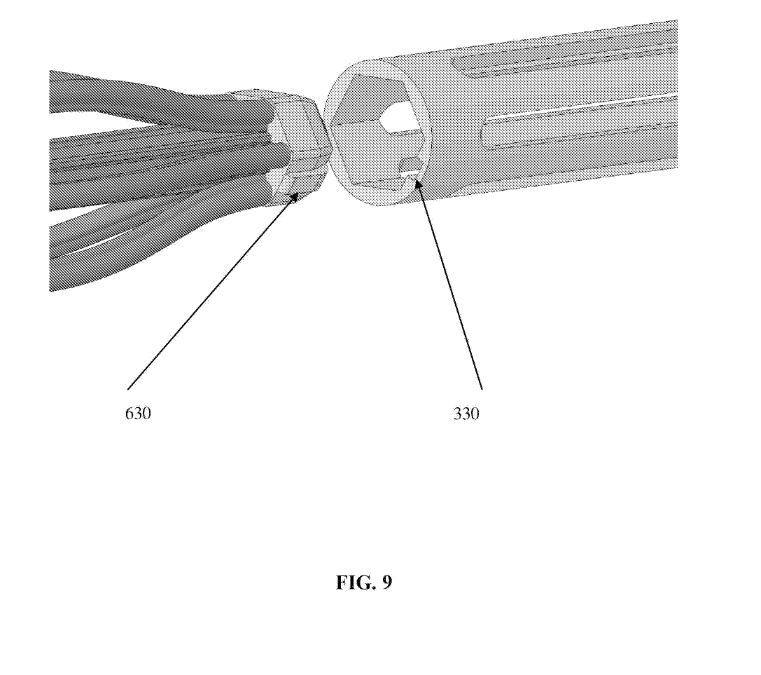

The expansion device may further include a keying feature or indexable feature 330, which mates the expansion device to the treatment device, so that the rotational angle of the treatment device remains constant with respect to the expansion device, thereby ensuring that the desired treatment program is achieved. The indexable feature 330 is further described in conjunction with FIG. 9 below.

FIG. 4 illustrates a deployment mechanism 400 for the expansion device that utilizes ratchet teeth. The outer expandable assembly while in its deflated or collapsed state may be inserted through an opening in the skin surface of the breast and into the cavity. Once in the cavity, the outer expandable assembly may be expanded or inflated until it substantially fills the cavity, before being locked or held in place by the deployment mechanism 400. An X-ray or CT scan may be used to determine when this point has been reached.

To expand the outer assembly in the expansion device, a movable plunger may be pushed forward or translated, thereby compressing the outer tubes of the outer assembly, thus causing them to bow or bend. The plunger may be moved until the cage defined by the outer tubes fills the cavity to a desired degree, such as until the outer cage substantially fills the cavity. The degree to which the outer cage defined by the outer tubes is expanded may vary to match different size cavities.

As seen in FIG. 4, the deployment mechanism 400 includes a series of ratchet teeth 410, which holds the outer assembly in place after the outer assembly has been deployed by pushing the plunger forward.

FIG. 5 illustrates another deployment mechanism 500 for the expansion device that utilizes a screw thread. The deployment mechanism 500 includes a lead screw 510. Turning the lead screw 510 causes the screw 510 to move forwards and backwards, in turn causing the outer tubes to expand or contract.

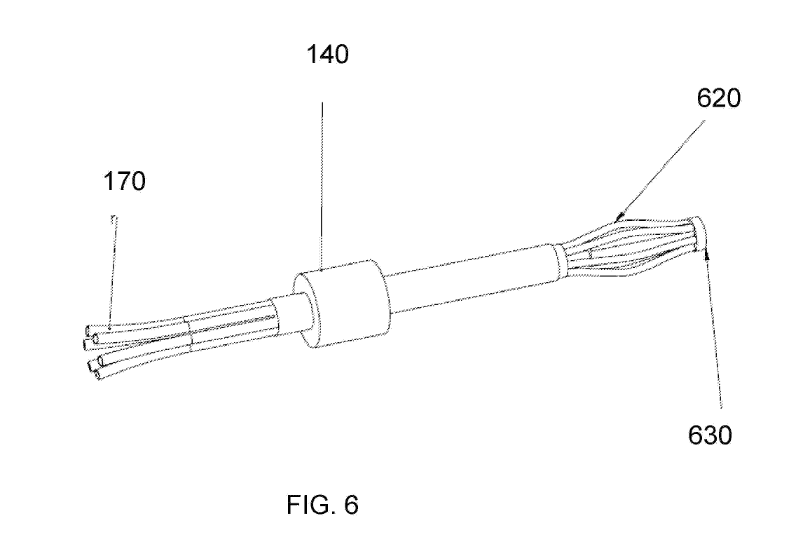

FIG. 6 illustrates various components of the treatment device. The treatment device includes inner expandable lumens or tubes 620, which may be used to guide the HDR radiation sources or seeds to desired locations as determined by the treatment plan. Connections 170 may be used to interface the inner lumens with the HDR afterloader. An indexable feature 630, further described in conjunction with FIG. 9 below, may be used to mate the treatment device to the expansion device.

The set of inner tubes 620 may include any number of tubes bundled together. The proximate ends of the inner tubes 620 may be bound together using any means, such as glue or an ultrasonic bond.

The inner tubes 620 may be made of any material, including material that is normally straight, but resiliently flexes after longitudinal compression. Plastic or any other type of material may be used. The inner tubes 620 may be of any number.

A central lumen may or may not be provided within one, some or all of the inner tubes 620. In some embodiments, the central lumen may be used as an access way for an additional treatment lumen, i.e. one or more radiation seeds may be inserted in the central lumen for some patients.

The outer tubes in the expansion device may be equal in number to the inner tubes 620. There may instead be more outer tubes than inner tubes or less.

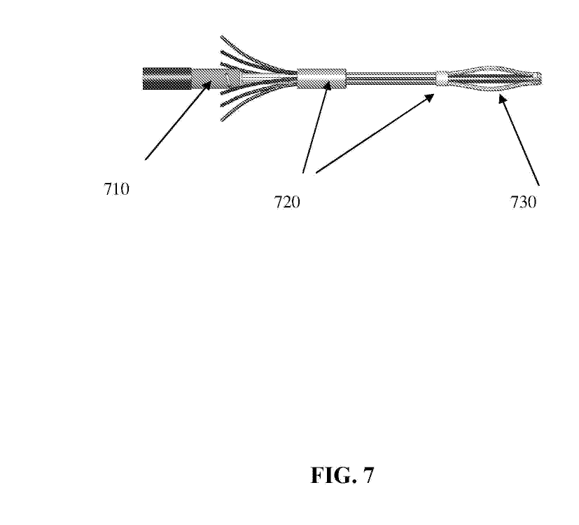

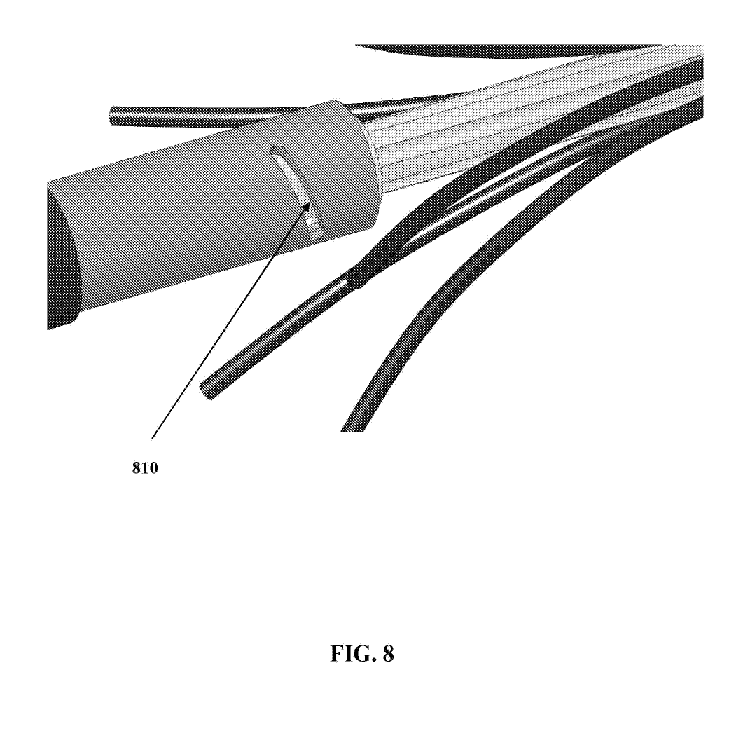

An actuator mechanism 140 may be used to expand and contract the inner lumens 620 of the inner assembly. The actuator mechanism 140 is further described in conjunction with FIGS. 7 and 8 below.

The inner tubes 620, as well as the outer tubes, may be expanded or contracted in unison. Alternatively, the expansion and contraction of each one of the inner tubes 620 or the outer tubes may be individually controllable.

The amount of bowing, bending, or flexing in each of the inner tubes 620 that results from the expansion and contraction caused by the actuator mechanism 140 may vary from procedure to procedure. The criteria that is employed for determining the amount may also vary.

During some procedures, all of the inner tubes 620 may initially be flexed in the same amount in accordance with predetermined information, such as a formula, algorithm, or specification. This predetermined information may be dependent upon the particular patient and his or her situation. Alternately, it may be patient independent.

FIG. 7 illustrates one example of an actuator mechanism for expanding and contracting the inner lumens in the treatment device. The actuator mechanism shown in FIG. 7 includes an actuator control 710 and a movable plunger 720. Twisting the actuator control 710 pushes the plunger 720 forward, causing the attached tubes 730 to expand. Twisting the actuator control 710 in the reverse direction causes the plunger 720 to move backwards, causing the attached tubes 730 to deflate or collapse.

After initially flexing each of the inner tubes to this predetermined amount, adjustments may be made to one or more of the inner tubes to protect healthy tissue and/or to increase the dose to certain areas of the cavity. For example, the bowing of one or more of the inner tubes may be individually reduced by moving its respective plunger backwards so as to better protect healthy tissue in its vicinity, such as skin, a lung or the heart. Similarly, the bowing of one or more of the inner tubes may be individually increased by moving its respective plunger forward.

FIG. 8 illustrates in more detail a pin and slot used in the actuator control 710 shown in FIG. 7. As seen in FIG. 8, the actuator control 710 includes a pin and slot, shown with reference numeral 810 in FIG. 8. When the actuator control 710 is twisted, the rotational force of the twisting motion is translated into a linear motion by means of the pin and slot 810.

FIG. 9 illustrates a keying feature for mating the treatment device to the expansion device, to achieve repeatable orientation. A keying feature 630 on the treatment device matches with the keying feature or slots 330 on the expansion device, to achieve constant and repeatable orientation. Only the mating portion of the expansion device is shown in FIG. 9, for clarity. The keying features 630 and 330 ensure that the user will always insert the treatment device at the same orientation relative to the expansion device, repeatedly.

The treatment device must be axially aligned with the expansion device. The inner assembly in the treatment device must not rotate with respect to the expansion device, because the positions of the inner lumens are fixedly determined by the treatment plan. The inner and outer cage or assembly must maintain their relative position and orientation with respect to each other, and not rotate with respect to each other, otherwise the treatment plan will be lost.

In one embodiment, a series of markers may be built into the outer or the inner lumens for location purposes and for planning purposes. In one embodiment, the markers may be rings. For example, in one exemplary embodiment, each one of the inner lumens may have a series of rings to mark the position of the lumen when the lumens are expanded. The markers or rings may be made of metallic, radio-opaque material, including without limitation tungsten. The ring material may have the ability to expand with the cage.

In operation, the outer expandable assembly 115, while in its deflated state, may be inserted through an opening in the skin surface of the treatment region (such as a breast, or a prostate) and into the post-surgical cavity. The opening and the pathway to the cavity may have been created during resection of a tumor or at any other time. Once in the cavity, the outer expandable assembly may be expanded using the actuator described above. The outer expandable assembly 115 may be expanded until it substantially fills the cavity. An X-ray or CT scan may be used to determine when this point has been reached.

Once the outer expandable assembly 115 has expanded to a desired level, for example expanded to conform to the post-surgical cavity, the deployment mechanism (shown as FIG. 4 or FIG. 5, for example) described above may be used to lock the outer expandable assembly 115 in place and maintain its deployment.

The inner expandable assembly 120 may then be inserted into the dry interior region created within the expandable sheath 320 of the outer expandable assembly 115, and expanded using the actuator 140 described above. The keying feature described above may be used, when inserting the inner assembly 120 within the expanded outer assembly, to ensure that the inner and the outer expandable assembly remain properly aligned.

The physician may use visual and other surgical aids to better assess the position of the inner tubes and/or the outer tubes inside the cavity. Such aids may be beneficial since the tubes may not be readily seen once they are inside the cavity. Examples of such visual or surgical aid include, but are not limited to ultrasound and CT.

Once the inner tubes and the outer tubes have been properly positioned, radioactive seeds, radioactive seed strands or other therapeutic or diagnostic elements may be placed into the tubes. In some embodiments, the therapeutic elements may be inserted only into the inner tubes. In other embodiments, at least some of the outer tubes may also be hollow lumens, and one or more therapeutic elements may be inserted into one or more of the outer tubes.

In some embodiments, surgical aids such as a CAT (computer aided tomography) scan may be used to determine whether the seed strands or other therapeutic elements have been accurately positioned in accordance with the radiation therapy plan, which may be created with surgical aids such as software designed to form an isodose profile. The appropriate isodose profile may call for the seeds to be inserted in a number of ways so as to vary the applied radiation level. For example, in some situations, the isodose profile may not require that any seed or other therapeutic element be inserted into one or more of the plurality of tubes. In some situations, two or more different seeds used on a single patient may have different activity levels so that some seeds are stronger than others.

After implantation, the inner tubes and/or the outer tubes may be cut. The cutting may be performed with a surgical instrument such as a scalpel. Alternatively, the inner and/or outer assembly may be self-cutting.

The inner assembly may be removed from the patient after a prescribed dose of radiation has been delivered. The outer assembly may remain, so that the inner assembly may be reinserted, or a different inner assembly inserted, or the outer assembly may also be removed, depending on the patient and the treatment plan.

The systems and methods described above may be used in conjunction with HDR (high dose radiation) or LDR (low dose radiation). In HDR, a very high dose radiation seed may be inserted, and the high dose radiation seed dwells in different places for finite time intervals, typically several minutes or so. In the LDR approach, lower dose radiation seeds may be inserted, usually for longer periods of time.

Different types of devices and apparatuses may be used for the tubes, to control and/or regulate their movement, and/or to feed them with radioactive seeds, than have been described. A radioactive source other than a seed may be used in addition or instead.

The patent applications that have been incorporated by reference in the Cross-Reference to Related Applications section of this application, as well as the patent that has been incorporated by reference in the Detailed Description section of this application, disclose a broad variety of related devices, components, and procedures. One or more of these may be used in conjunction with the devices, components, and/or procedures that are described in this application and/or in lieu of some or all of them.

Nothing that has been stated or illustrated is intended to cause a dedication of any component, step, feature, object, benefit, advantage, or equivalent to the public.

What is claimed is:

1. A brachytherapy device comprising:
an expansion device having an outer expandable assembly that is configured to be implanted into a post-surgical cavity;
a treatment device having an inner expandable assembly that is mated to the expansion device during treatment fractions and a pathway for a radiation source.
